SEQUENCE LISTING (6 sequences)

SEQ ID NO: 1 - protein, 1260 aa, Severe acute respiratory syndrome coronavirus 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 4 aa, unidentified
RRAR

SEQ ID NO: 3 - protein, 4 aa, unidentified
QQAQ

SEQ ID NO: 4 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1256 aa, synthetic construct
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1269 aa, synthetic construct
MFVFLVLLPLVSSQCVMPLFNLITTTQSYTNSFTRGVYYPDKVFRSSVLHLTQDLFLPFFSNVTWFHAISGTNGTKRFDNPVLPFNDGVYFASTEKSNIIRGWIFGTTLDSKTQSLLIVNNATNVFIKVCEFQFCNDPFLDVYHKNNKSWMESESGVYSSANNCTFEYVSQPFLMDLEGKQGNFKNLREFVFKNIDGYFKIYSKHTPIIGRDFPQGFSALEPLVDLPIGINITRFQTLLALNRSYLTPGDSSSGWTAGAADYYVGYLQPRTFLLKYNENGTITDAVDCALDPLSETKCTLKSFTVEKGIYQTSNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGVCASYQTQTKSRQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2026/0259196 A1
(43) Pub. Date: Sep. 3, 2026

(54) ASSAYS FOR THE EVALUATION OF SEASONAL COVID-INFLUENZA COMBINATION VACCINES

(71) Applicant: NOVAVAX, INC., Gaithersburg, MD (US)

(72) Inventors: Raj KALKERI, Gaithersburg, MD (US); Mingzhu ZHU, Gaithersburg, MD (US); Joyce S. PLESTED, Gaithersburg, MD (US); Timothy VINCENT, Gaithersburg, MD (US); Wendy Shane CLONEY-CLARK, Gaithersburg, MD (US); Zhaohui CAI, Gaithersburg, MD (US)

(21) Appl. No.: 19/533,966

(22) Filed: Feb. 9, 2026

Related U.S. Application Data
(60) Provisional application No. 63/755,836, filed on Feb. 7, 2025.

(51) Int. Cl. G01N 33/50; G01N 33/569

(52) U.S. Cl. CPC G01N 33/5023; G01N 33/502; G01N 33/5047; G01N 33/56983; G01N 2333/11; G01N 2333/15; G01N 2333/165; G01N 2333/948; G01N 2469/20

(57) ABSTRACT

Disclosed are methods for detecting if a biological sample (e.g., saliva, serum, blood, plasma) from a subject that has been administered a an immunogenic composition directed to a seasonal influenza virus and an immunogenic composition directed to a SARS-COV-2 virus contains antibodies that inhibit influenza virus hemagglutinin (HA) glycoprotein hemagglutination activity, antibodies that neutralize SARS-COV-2 virus, antibodies that bind SARS-COV-2 spike protein, and/or antibodies that neutralize seasonal influenza virus. The methods include assaying a biological sample, where the biological sample is from a subject that has been administered an immunogenic composition directed to a seasonal influenza virus and an immunogenic composition directed to a SARS-COV-2 virus, and is not significantly affected by interference of analyte detection between the methods.

### RELATED APPLICATION

The present application claims priority to U.S. Provisional Patent Application No. 63/755,836, filed on Feb. 7, 2025, the contents of which is incorporated by reference in its entirety.

### REFERENCE TO AN ELECTRONIC SEQUENCE LISTING

This application contains a Sequence Listing which has been submitted electronically in XML format and is hereby incorporated by reference in its entirety. Said XML copy, created on Feb. 9, 2026, is named 1450_130US1_Sequence_Listing_02_09_2026 and is 11,341 bytes in size.

### FIELD OF THE INVENTION

The present disclosure is generally related to methods for evaluating efficacy of a seasonal COVID-influenza combination vaccine.

### BACKGROUND OF THE INVENTION

Seasonal influenza virus infections pose a significant threat to public health globally, resulting in 3-5 million cases of severe illness and 290,000-650,000 deaths, annually. Annual vaccination is recommended as the most effective approach for the prevention and control of seasonal influenza. However, currently available seasonal influenza vaccines confer variable protection due to antigenic changes resulting from the accumulation of diverse mutations.

The viral hemagglutinin (HA) surface glycoproteins are key determinants of vaccine efficacy against seasonal circulating strains of influenza. The traditional hemagglutination inhibition (HAI) assay is considered the gold standard method for determining an influenza vaccine-elicited immune response and quantitative antibody titers for the influenza virus. However, new influenza vaccine development is challenging due, in part, to traditional, labor-intensive, and cumbersome HAI assay methods involving avian red blood cells (RBCs), complex sample preparation, subjectivity, and low assay throughput. In addition, certain clades of A/H3N2 viruses agglutinate avian RBCs poorly and alternative assays, such as virus neutralization must be used.

The coronavirus disease 2019 (COVID-19) pandemic is caused by severe acute respiratory syndrome coronavirus 2 (SARS-COV-2). The emergence of variants (such as Alpha, Beta, Gamma, Delta, and multiple Omicron subvariants) has led to ongoing transmission of the virus. Some SARS-COV-2 variants (such as Omicron) have immune evasion properties, thus reducing the effectiveness of COVID-19 vaccines. There is a need for additional correlates of protection (CoPs) for COVID vaccine efficacy to help track immune evasion and understand the needs of the vaccine development landscape (e.g., development of new vaccines based on variant S protein sequences). Validated CoPs help to extrapolate vaccine efficacy/immunogenicity results to populations or formulations/schedules not represented in clinical trials. Of particular interest are CoPs for durability and level of vaccine-driven protection against ancestral and variant strains.

Biomarkers of immunogenicity, which may eventually be proven to be correlates of protection, are critical for assessment of vaccines. In particular, the amount of anti-SARS-COV-2 S (spike) antibodies in a biological sample determines the effectiveness of the humoral response induced by the vaccine.

Given the seasonality trend of SARS-COV-2 and the seasonality of influenza virus, vaccines that target both viruses are being developed. Due to the possibility of potential interference with the analyte detection between assays that evaluate immune response to SARS-COV-2 vaccines and seasonal influenza virus vaccines, there is a need for developing and validating methods suitable for evaluation of biological samples from subjects administered one or more immunogenic compositions comprising an antigen to each of SARS-CoV-2 and seasonal influenza virus.

### SUMMARY OF THE INVENTION

The present disclosure provides methods for evaluating antibody response in subjects administered one or more immunogenic composition comprising a SARS-COV-2 antigen and a seasonal influenza antigen.
In Aspect 1, a method for determining if a biological sample contains antibodies that inhibit influenza virus hemagglutinin (HA) glycoprotein hemagglutination activity is provided. The method includes (a) obtaining the biological sample, where the biological sample is from a subject that has been simultaneously administered an immunogenic composition directed to a seasonal influenza virus and an immunogenic composition directed to a SARS-COV-2 virus; (b) reducing or removing non-specific inhibitors of HA from the biological sample to produce a treated sample; (c) exposing the treated sample to an influenza virus having an HA glycoprotein to produce a sample-virus mixture; (d) incubating the sample-virus mixture with human red blood cells (RBCs), the human RBCs being at a concentration of about 0.5% to about 1%, or about 0.75%; and (e) detecting agglutination of the human RBCs; where the biological sample contains antibodies that inhibit HA glycoprotein hemagglutination activity if agglutination of the RBCs is not detected.
In Aspect 2, the biological sample in the method of Aspect 1 can be serially diluted, and each serial dilution of the sample is subjected to steps (b), (c), (d), and (e).
In Aspect 3, the method of Aspect 2 can include determining the hemagglutination inhibition titer of the biological sample.
In Aspect 4, the virus used in the method of any one of Aspects 1-3 can comprise an egg-derived influenza virus, or a virus-like particle (VLP).
In Aspect 5, a method for determining if a biological sample contains antibodies that neutralize a SARS-COV-2 virus is provided. The method includes (a) obtaining the biological sample, where the biological sample is from a subject that has been simultaneously administered an immunogenic composition directed to a seasonal influenza virus and an immunogenic composition directed to a SARS-COV-2 virus; (b) contacting the biological sample with a replication-deficient Maloney murine leukemia virus (MLV) pseudovirus to form a sample-pseudovirus mixture, the MLV pseudovirus comprising a recombinant SARS-CoV-2 S (rS) glycoprotein and a gene of interest (GOI) encoding a reporter protein; (c) adding cells expressing angiotensin converting enzyme 2 (ACE2) to the sample-pseudovirus mixture, where the cells are A549 or HEK293T cells; (d) incubating the sample-pseudovirus mixture with the cells expressing ACE2 at 37° C., and quantifying expression of the reporter protein in the cells expressing ACE2 at 48 hours of incubation; (e) contacting control A549 or HEK293T cells expressing ACE2 with the MLV pseudovirus to form a virus-only control, where the MLV pseudovirus has not been contacted with the biological sample; and (f) incubating the virus-only control with the control cells at 37° C., and quantifying expression of the reporter protein in the control cells expressing ACE2 at 48 hours of incubation; where if the expression of the reporter protein in the cells of step (d) is less than expression of the reporter protein in the control cells of step (f), then the biological sample contains neutralizing antibodies against the SARS-COV-2 virus.
In Aspect 6, a method for simultaneously determining if a biological sample contains neutralizing antibodies against two or more SARS-COV-2 virus strains is provided. The method includes (a) obtaining the biological sample, where the biological sample is from a subject that has been simultaneously administered an immunogenic composition directed to a seasonal influenza virus and an immunogenic composition directed to a SARS-COV-2 virus; (b) contacting the biological sample with a first replication-deficient MLV pseudovirus and a second replication-deficient MLV pseudovirus to form a sample-pseudovirus mixture, the first MLV pseudovirus comprising an rS glycoprotein from a first SARS-COV-2 virus strain and a GOI encoding a first reporter protein, and the second MLV pseudovirus comprising an rS glycoprotein from a second SARS-COV-2 virus strain and a GOI encoding a second reporter protein; (c) adding cells expressing ACE2 to the sample-pseudovirus mixture, where the cells are A549 or HEK293T cells; (d) incubating the sample-pseudovirus mixture with the cells expressing ACE2 at 37° C., and quantifying expression of the first reporter protein and the second reporter protein in the cells expressing ACE2 at 48 hours of incubation; (e) contacting control A549 or HEK293T cells expressing ACE2 with the first MLV pseudovirus and the second MLV pseudovirus to form a virus-only control, where the first MLV pseudovirus and the second MLV pseudovirus have not been contacted with the biological sample; and (f) incubating the virus-only control with the control cells at 37° C., and quantifying expression of the first reporter protein and the second reporter protein in the control cells expressing ACE2 at 48 hours of incubation; where if the expression of the first reporter protein in the cells of step (d) is less than expression of the first reporter protein in the control cells of step (f), then the biological sample contains neutralizing antibodies against the first SARS-COV-2 virus strain, and where if the expression of the second reporter protein in the cells of step (d) is less than expression of the second reporter protein in the control cells of step (f), then the biological sample contains neutralizing antibodies against the second SARS-COV-2 virus strain.
In Aspect 7, the first reporter protein used in the method of Aspect 6 can be red fluorescent protein (RFP) and the second reporter protein is green fluorescent protein (GFP).
In Aspect 8, a method for determining if a biological sample contains antibodies that bind to a SARS-COV-2 Spike(S) glycoprotein is provided. The method includes (a) obtaining the biological sample, where the biological sample is from a subject that has been simultaneously administered an immunogenic composition directed to a seasonal influenza virus and an immunogenic composition directed to a SARS-COV-2 virus; (b) providing a surface coated with a recombinant SARS-COV-2 S (rS) glycoprotein; (c) exposing the surface to the biological sample; (d) exposing the surface to a secondary antibody; and (d) detecting the secondary antibody that is bound to the surface; where the biological sample contains antibodies that bind to the rS glycoprotein if the secondary antibody is detected.
In Aspect 9, the rS glycoprotein on the coated surface in the method of Aspect 8 can have at least 90% identity to SEQ ID NO: 1.
In Aspect 10, the rS glycoprotein on the coated surface in the method of Aspect 8 or Aspect 9 can comprise an inactive furin cleavage site.
In Aspect 11, the rS glycoprotein on the coated surface in the method of any one of Aspects 8-10 can have an amino acid sequence where amino acids 973 and 974 of the SARS-CoV-2 S glycoprotein are proline, as compared to a wild-type SARS-COV-2 S glycoprotein having the amino acid sequence of SEQ ID NO: 1.
In Aspect 12, the antibodies that bind to the rS glycoprotein on the coated surface in the method of any one of Aspects 8-11 can comprise IgG.
In Aspect 13, the secondary antibody in the method of any one of Aspects 8-11 can comprise an anti-IgG antibody.
In Aspect 14, the immunogenic composition directed to the seasonal influenza virus and the immunogenic composition directed to the SARS-COV-2 virus in the method of any one of Aspects 1-14 can comprise a single immunogenic composition.
In Aspect 15, the immunogenic composition directed to the seasonal influenza virus in the method of any one of Aspects 1-14 can comprise an antigen from each of at least three different seasonal influenza viruses.
In Aspect 16, the immunogenic composition directed to the seasonal influenza virus in the method of Aspect 15 can comprise an antigen from each of at four different seasonal influenza viruses.
In Aspect 17, the immunogenic composition directed to the seasonal influenza virus in the method of Aspect 15 or 16 can comprise an antigen from each of at least one Type A influenza virus and at least one Type B influenza virus.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110. When applied to a range, “about” indicates the lower value of the range minus 10%, and the upper value of the range plus 10%. For example, “a range of about 100 to 200” encompasses a range of 90 (lower value-10%) to 220 (upper value+10%).

As used herein, “substantially free” refers to exclusion of a substance (e.g. a compound, polynucleotide, or polypeptide) such that the substance forms the minority percent (e.g., less than 10%, less than 5%, less than 1%, or less than 0.1%) of the sample in which it is contained.

As used herein, the term “influenza virus” used in reference to an hemagglutination inhibition (HAI) assay provided herein refers to a live, modified live (e.g., weakened), or killed influenza virus (e.g., an egg-derived influenza virus) or a virus-like particle comprising an influenza virus hemagglutinin (HA) glycoprotein. An influenza virus herein includes a functional HA glycoprotein. In some embodiments, an influenza virus can include an influenza virus neuraminidase (NA) glycoprotein. In some embodiments, an influenza virus can lack a functional NA glycoprotein (e.g., having no NA glycoprotein, or having an NA glycoprotein without enzymatic function).

As used herein, the term “hemagglutination activity” refers to the process of agglutinating red blood cells (RBCs). “Agglutination” refers to visible clumping together of RBCs. While other molecules can cause agglutination of RBCs, as used herein, hemagglutination activity refers to agglutination of RBCs mediated by influenza virus HA glycoprotein. “Non-specific hemagglutination activity” refers herein to agglutination of RBCs mediated by a factor other than an influenza virus HA (e.g., NA glycoprotein-mediated agglutination).

As used herein, the term “hemagglutination inhibition” (“HAI”) or “inhibition of influenza virus HA glycoprotein hemagglutination activity” refers to a reduction or elimination of hemagglutination activity mediated by influenza virus HA glycoprotein. A new HAI assay described herein can be used to detect and/or quantitate antibodies in a biological sample that can inhibit influenza virus HA protein hemagglutination activity.

As used herein, the term “wild-type” refers to a naturally occurring virus or the amino acid sequence of a protein or glycoprotein of a naturally occurring virus. For example, a wild-type SARS-COV-2 virus includes any naturally occurring strain, such as the Wuhan SARS-COV-2 strain, a B.1.1.7 SARS-COV-2 strain; a B.1.351 SARS-COV-2 strain; a P. 1 SARS-CoV-2 strain; a Cal.20C SARS-COV-2 strain; a B.1.617.2 SARS-COV-2 strain; a B.1.525 SARS-COV-2 strain; a B.1.526 SARS-COV-2 strain; a B.1.617.1; SARS-COV-2 strain; a C.37 SARS-COV-2 strain; aB.1.621 SARS-COV-2 strain; a B.1.1.529 SARS-COV-2 strain, or the like. Similarly, a wild-type SARS-COV-2 spike(S) glycoprotein sequence comprises a naturally occurring S glycoprotein from any naturally occurring SARS-COV-2 strain.

As used herein, the term “modified” referring to a virus or viral protein or glycoprotein refers to a virus or protein/glycoprotein that differs from a naturally occurring virus or protein/glycoprotein. A modified protein or glycoprotein can have an amino acid sequence that is mutated relative to an amino acid sequence of a protein or glycoprotein from a naturally occurring virus. For example, a modified SARS-COV-2 S glycoprotein has an amino acid sequence that is less than 100% identical to a wild-type SARS-COV-2 S glycoprotein. In another example, a modified SARS-COV-2 S glycoprotein can be a stabilized SARS-COV-2 S glycoprotein having an inactive furin cleavage site and prolines at each of amino acids 973 and 974 when aligned and numbered relative to SEQ ID NO: 1. In another example, a modified virus can comprise a pseudovirus, a virus-like particle, or an egg-derived virus.

As used herein, a “receptor-destroying enzyme” (“RDE”) refers to an enzyme that cleaves sialic acid from glycoproteins and glycolipids.

As used herein, the term “heat-inactivated” or “heat-inactivation” refers to the treatment of a sample with sufficient heat to reduce or remove enzymatic activity (e.g., RDE enzymatic activity), but retain antibody function in the biological sample. Heat-inactivation can be performed at any suitable temperature and time. For example, a biological sample can be heat inactivated at a temperature of about 50° C. to about 60° C. (e.g., about 54° C. to about 58° C.) for about 20 minutes to about 40 minutes (e.g., about 25 minutes to about 35 minutes, or about 28 minutes to about 32 minutes).

As use herein, the term “hemagglutination inhibition titer” (“HAI titer”) refers to the highest tested dilution of a biological sample that achieves hemagglutination inhibition. The HAI titer is generally reported as the dilution factor. For example, if the highest dilution of a sample that achieves hemagglutination inhibition is 1:10, then the HAI titer is 10.

As used herein, a “hemagglutination unit” (“HAg unit”) refers to the lowest concentration of influenza virus that fully agglutinates a 0.75% suspension of human RBCs in Dulbecco's phosphate buffered saline (DPBS). Generally, to determine the concentration of influenza virus that fully agglutinates a 0.75% suspension of human RBCs, serial dilutions of influenza virus are combined with the RBC suspension to determine the lowest concentration that achieves complete agglutination of the RBCs. 4 HAg units is thus an amount of virus that is four times that of a single HAg.

As used herein, an “immunogenic composition” is a composition that comprises an antigen where administration of the composition to a subject results in the development in the subject of a humoral and/or a cellular immune response to the antigen.

As used herein, the term “adjuvant” refers to a compound or substance that, when used in combination with an antigen, augments or otherwise alters or modifies the immune response induced against the antigen. Modification of the immune response may include intensification or broadening the specificity of either or both antibody and cellular immune responses.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an antigen derived from a pathogen, which is used to induce an immune response against the pathogen. The immune response may include formation of antibodies and/or a cell-mediated response. Depending on context, the term “vaccine” may also refer to a suspension or solution of an antigen that is administered to a subject to produce an immune response. Preferably, a vaccine induces an immune response that is effective at preventing infection from one or more seasonal influenza virus strain.

As used herein, the terms “simultaneous” or “simultaneously” in context of administration of an immunogenic composition refers to administration of an immunogenic composition that includes a SARS-COV-2 antigen and an immunogenic composition that includes a seasonal influenza antigen within 7 days of each other, preferably within 1 day of each other, or most preferably at the same time. In some embodiments, the immunogenic composition that includes a SARS-COV-2 antigen and the immunogenic composition that includes a seasonal influenza antigen are the same immunogenic composition. That is, a single immunogenic composition can include both a SARS-COV-2 antigen and a seasonal influenza antigen. In some embodiments, a single immunogenic composition can comprise 2 or more SARS-COV-2 antigens and/or 2 or more seasonal influenza antigens. For example, a single immunogenic composition can comprise a SARS-COV-2 antigen (e.g., a recombinant SARS-CoV-2 spike (rS) glycoprotein) and an antigen (e.g., a hemagglutinin (HA) glycoprotein) from 2, 3, or 4 different seasonal influenza strains.

As used herein, the terms “simultaneous” or “simultaneously” in context of determining whether a biological sample contains antibodies of interest (e.g., IgG, neutralizing antibodies, or the like) refers to determining the presence of two or more types of antibodies in a single assay. For example, a method for simultaneously determining if a biological sample contains neutralizing antibodies against two or more SARS-COV-2 virus strains means that the method detects neutralizing antibodies against each of two ore more SARS-COV-2 virus strains in the same assay.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject can be up to 4 months old, or up to 6 months old. In aspects, the adults can be seniors about 65 years or older, or about 60 years or older. In aspects, the subject can be a pregnant woman or a woman intending to become pregnant. In other aspects, subject can be not a human, such as a non-human primate, for example, a baboon, a chimpanzee, a gorilla, or a macaque. In certain aspects, the subject may be a pet, such as a dog or cat, or livestock, such as poultry or cattle.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

### Methods of Detecting Anti-Influenza HA Antibodies

The viral hemagglutinin (HA) surface glycoproteins are key determinants of vaccine efficacy against seasonal circulating strains of influenza. The hemagglutination inhibition (HAI) assay represents the gold standard method for determining an influenza vaccine-elicited immune response and quantitative antibody titers for the influenza virus [7-9]. However, new influenza vaccine development remains challenging due, in part, to traditional, labor-intensive, and cumbersome HAI assay methods involving avian red blood cells (RBCs), complex sample preparation, subjectivity, and low assay throughput. In addition, certain clades of A/H3N2 viruses agglutinate avian RBCs poorly and alternative assays such as virus neutralization assays must be used. A modified HAI assay protocol has been developed to generate more reliable and consistent data using a 0.08% solution of stabilized human RBCs and an assay buffer containing bovine serum albumin (Morokutti, A.; Redlberger-fritz, M.; Nakowitsch, S.; Krenn, B. M.; Wressnigg, N. Validation of the Modified Hemagglutination Inhibition Assay (MHAI), a Robust and Sensitive Serological Test for Analysis of Influenza Virus-Specific Immune Response. J. Clin. Virol. 2013, 56, 323-330.), avoiding partial hemagglutination or irregular shapes of RBC precipitation with avian-origin RBCs. An automated CypherOne™ Hemagglutination Analyzer (InDevR, Boulder, CO, USA) has also been used to determine and provide a visual permanent record of assay results to eliminate the need for tilting of the HAI plate before image interpretation, thereby simplifying the assay and reducing assay turnaround time (Wilson, G.; Ye, Z.; Xie, H.; Vahl, S.; Dawson, E.; Rowlen, K. Automated Interpretation of Influenza Hemagglutination Inhibition (HAI) Assay: Is Plate Tilting Necessary? PLOS ONE 2017, 12, e0179939.). A recent report employed a combination of guinea pig RBCs (instead of avian RBCs), neuraminidase (NA) inhibitor oseltamivir (to prevent NA-mediated agglutination), and CypherOne™ (InDevR, Boulder, CO, USA) HAI plate reader-based automation but focused solely on the HAI assay for H3N2 influenza strains (Sawant, S.; Gurley, S. A.; Overman, R. G.; Sharak, A.; Mudrak, S. V.; Oguiniii, T.; Sempowski, G. D.; Sarzotti-kelsoe, M.; Walter, E. B.; Xie, H.; et al. H3N2 in Fluenza Hemagglutination Inhibition Method Qualification with Data Driven Statistical Methods for Human Clinical Trials. Front. Immunol. 2023, 14, 1155880.). Although all these studies have reported reproducible and reliable results, they do not achieve a single, harmonized high-throughput HAI assay method for both the H1N1 and H3N2 strains as well as B-type viruses.

Provided herein is an improved and objective novel HAI assay that was developed with recombinant virus-like particles (VLPs) or egg-derived virus as agglutinins and human red blood cells (hemagglutination indicator particle) for the detection and/or measurement of anti-influenza HA antibody titers in biological samples (e.g., human serum). In some embodiments, an HAI assay described herein can include the inactivation of neuraminidase-mediated RBC agglutination, e.g., using oseltamivir treatment. In some embodiments, an HAI assay described herein can include an automated image reader-based analysis of hemagglutination. An HAI assay provided herein can have one or more benefits over previously described HAI assays, as evidenced by HAI assay validation described herein. For example, validated HAI results were equivalent for single and duplicate sample testing and correlated well with a qualified live wild-type influenza microneutralization assay, which demonstrate the suitability of this high-throughput novel modified validated HAI assay for evaluating vaccine immunogenicity and efficacy. In addition, the described HAI assay can be used on singleton samples with similar accuracy to duplicate samples, which can provide a benefit of reducing the biological sample volume, reducing laboratory resources, and/or reduce costs needed to analyze HAI titer in a biological sample. In some embodiments, an HAI assay provided herein can also provide a benefit of being able to be performed in a standardized manner for many different influenza strains.

The herein described assay HAI assay was demonstrated to be precise as indicated by the percent geometric coefficient of variation for intra-, inter-, and total assay precision, as well as accurate as evidenced by percent bias measurements. The herein described HAI assay exhibited linearity, specificity for homologous type/subtype strains, and sensitivity with a starting dilution of 1:10, although other starting dilutions may be suitable. Assay robustness and sample stability were demonstrated as a percentage difference compared to reference condition.

Provided herein are methods for determining if a biological sample (e.g., human serum) contains antibodies that inhibit influenza virus HA glycoprotein hemagglutination activity, comprising: (i) obtaining a biological sample from a subject that has been simultaneously administered an immunogenic composition directed to a seasonal influenza virus and an immunogenic composition directed to a SARS-COV-2 virus; (ii) reducing or removing non-specific inhibitors of HA from the biological sample to produce a treated sample; (iii) exposing the treated sample to an influenza virus having an HA glycoprotein to produce a sample-virus mixture; (iv) incubating the sample-virus mixture with human red blood cells (RBCs); and (v) detecting agglutination of the human RBCs; wherein the biological sample contains antibodies that inhibit HA glycoprotein hemagglutination activity if agglutination of the RBCs is not detected.

In some embodiments, a biological sample can be treated with a receptor-destroying enzyme (RDE) to reduce or remove non-specific inhibitors of HA. A biological sample can be incubated with an RDE at a time and temperature sufficient to reduce or remove non-specific inhibitors of HA. For example, a biological sample can be incubated with an RDE at about 37° C. for about 15 hours to about 24 hours (e.g., about 18 hours to about 20 hours). In some embodiments, after being used to reduce or remove non-specific inhibitors of HA, RDE can be heat-inactivated, e.g., at a temperature of about 50° C. to about 60° C. (e.g., about 54° C. to about 58° C.) for about 20 minutes to about 40 minutes (e.g., about 25 minutes to about 35 minutes, or about 28 minutes to about 32 minutes).

In some embodiments, a treated biological sample can be stored prior to use in an HAI assay described herein. A treated biological sample can be stored for up to 2 months (e.g., up to 1 month) at 2° C. to 8° C. before use in an HAI assay without significantly impacting assay results. In some embodiments, a treated biological sample can be stored for up to 2 months at ≤−20° C. (e.g., up to 1 month) before use in an HAI assay without significantly impacting assay results. Surprisingly, even with two freeze/thaw cycles, treated biological samples can be stored for up to 2 months at ≤−20° C. before use in an HAI assay without significantly impacting assay results. In some embodiments, a biological sample that is undiluted (e.g., untreated), can be stored at ≤−20°=10° C. for at least 1 month, even with seven freeze/thaw cycles, before use in an HAI assay without significantly impacting assay results. A significant impact on assay results is generally considered to be more than a 2-fold difference in results relative to a control—in this case, results using a treated biological sample that has been stored for less than 7 days. In some embodiments, the amount of time that biological sample can be stored before use can depend on the influenza virus being used in the assay.

In some embodiments, a biological sample can be diluted, either before or after treatment to reduce or remove non-specific inhibitors of HA, prior to further assay steps. For example, a biological sample can be diluted to reflect a predetermined hemagglutination inhibition titer threshold (e.g., 1:2, 1:4, 1:5, 1:10, or the like). In some embodiments, a biological sample can be serially diluted two or more times (e.g., 2 to 50 times, 2 to 20 times, 2 to 15 times, 3, 4, 5, 6, 7, 8, 9, 10, 11, or 12 times). Serial dilution can be performed at any appropriate ratio (e.g., 1:2, 1:3, 1:4, 1:10, and the like). In some embodiments, a biological sample can first be diluted one or more times to reflect a predetermined hemagglutination inhibition titer threshold (e.g., 1:4, 1:10, or the like), and then be further serially diluted (e.g., 1:2, 1:4, or the like). In some embodiments, a biological sample can be diluted in a buffer, such as Dulbeco's phosphate buffered saline (DPBS), optionally with calcium and/or magnesium.

A treated biological sample, and optionally, dilutions of the treated biological sample, is exposed to an influenza virus having an HA glycoprotein to produce a sample-virus mixture. The influenza virus acts an agglutinin of RBCs. In some embodiments, an influenza virus can be an egg-derived influenza virus. Such an egg-derived virus is typically a weakened virus. Any available egg-derived influenza virus can be used, including, for example, one or more of an A/H3N2 virus (e.g., A/Kansas/14/2017, A/Coatia/10136RV/2023, or the like), an A/H1N1 virus (e.g., A/Brisbane/02/2018, A/Victoria/4897/2022, or the like), a B/Victoria lineage virus (e.g., B/Maryland/15/2016, B/Austria/1359417/2021, or the like), and a B/Yamagata lineage virus (e.g., B/Phuket/3073/2013). Other suitable egg-derived influenza viruses can be any influenza A virus, having any hemagglutinin subtype (H1-H18) and any neuraminidase subtype (N1-N11), or any influenza B virus, from any lineage.

In some embodiments, an influenza virus can be a virus-like particle (VLP). A VLP can be produced in insect cells (e.g., Sf9) infected with baculoviruses bearing cloned genes of influenza HA glycoprotein, NA glycoprotein, and influenza matrix protein (M1). Any suitable HA glycoprotein, NA glycoprotein, and M1 protein can be used. Beneficially, an influenza virus comprising a VLP used in an HAI assay described herein can be used to assess the presence and/or titer of antibodies that inhibit HA glycoprotein hemagglutination activity for both VLPs that are analogous to egg-derived influenza virus strains, as wells as VLPs that are analogous to wild type strains for which there are no analogous egg-derived influenza virus strains. Suitable VLPs can include, for example, VLP analogues of one or more of an A/H3N2 virus (e.g., A/Kansas/14/2017, A/Coatia/10136RV/2023, A/California/94/2019, A/Cardiff/0508/2019, A/Netherlands/1268/2019, A/Tokyo/EH1801/2018, or the like), an A/H1N1 virus (e.g., A/Brisbane/02/2018, A/Victoria/4897/2022, or the like), a B/Victoria lineage virus (e.g., B/Maryland/15/2016, B/Austria/1359417/2021, or the like), and a B/Yamagata lineage virus (e.g., B/Phuket/3073/2013). Other suitable VLPs can be analogous to any influenza A virus, having any hemagglutinin subtype (H1-H18) and any neuraminidase subtype (N1-N11), or any influenza B virus, from any lineage. A method of making such a VLP can be found in Smith, et al. (Development of influenza H7N9 virus like particle (VLP) vaccine: Homologous A/Anhui/1/2013 (H7N9) protection and heterologous A/chicken/Jalisco/CPA1/2012 (H7N3) cross-protection in vaccinated mice challenged with H7N9 virus. Vaccine 2013, 31, 4305-4313).

In some embodiments, an influenza virus (egg-derived or VLP) can be treated with an NA glycoprotein inhibitor, such as oseltamivir, to reduce or prevent NA glycoprotein-mediated RBC agglutination. In some embodiments, oseltamivir can be included in a stock composition of influenza virus at a concentration of about 60 nM to about 100 nM, or about 80 nM. However, in some embodiments, a VLP can exclude a functional NA glycoprotein. That is, in some embodiments, a VLP can have a NA glycoprotein that is enzymatically inactive or have no NA glycoprotein included.

A stock comprising influenza virus can be diluted to achieve about 2 to about 10 (e.g., about 3 to about 5, or about 4) hemagglutination (HAg) units when combined with biological sample to form a sample-virus mixture. In some embodiments, a stock comprising influenza virus can be diluted in a buffer, such as DPBS.

A sample-virus mixture is incubated with human red blood cells (RBCs) prior to detecting agglutination of the RBCs. The human RBCs are preferably in suspension at a concentration of about 0.5% to about 1% (e.g., about 0.75%). Incubation can be done under conditions that promote hemagglutination in the absence of hemagglutination inhibitors. For example, incubation can be performed at a temperature of from about 15° C. to about 30° C. (e.g., about 18° C. to about 28° C., or about 20° C. to about 22° C.) for about 40 minutes to about 80 minutes (e.g., about 50 minutes to about 70 minutes, or about 60 minutes).

Preferably, human RBCs used in an HAI assay provided herein are from Type O blood. In some embodiments, human RBCs can be treated with an anticoagulant, such as K3EDTA. In some embodiments, human RBCs can be tested to ensure that they agglutinate similarly to previous lots of human RBCs prior to use. For example, RBCs can be tested in an HAI assay using previously tested biological sample(s) (e.g., human serum) to ensure that the results are similar (e.g., within 2-fold) to previous lots of human RBCs. In some embodiments, human RBCs can be tested with negative and/or positive control biological sample(s). A positive control biological sample can comprise, e.g., sheep serum from HA-hyperimmune sheep, human serum that previously showed positive HAI in a previous HAI assay, or the like. A negative control biological sample can comprise, e.g., anti-HA antibody depleted serum (e.g., from a human).

In some embodiments, a human RBC suspension can be stored prior to use in an HAI assay described herein. A human RBC suspension can be stored for up to 2 weeks (e.g., up to 10 days, or up to 1 week) at 2° C. to 8° C. before use in an HAI assay without significantly impacting assay results. A significant impact on assay results is generally considered to be more than a 2-fold difference in results relative to a control—in this case, results using a human RBC suspension that has been used immediately after dilution of RBCs to form the suspension. In some embodiments, the amount of time that a human RBC suspension can be stored before use can depend on the influenza virus being used in the assay.

Following incubation of human RBCs with sample-virus mixture, agglutination of the RBCs is assessed. Preferably, assessment of agglutination is performed 0 to about 90 minutes (e.g., 0 to about 60 minutes, about 15 to about 45 minutes, or about 30 minutes) following the end of incubation, e.g., at about 75 minutes to about 150 minutes, about 80 minutes to about 100 minutes, or about 90 minutes after incubation of human RBCs with sample-virus mixture begins.

Agglutination of human RBCs can be assessed manually, or using an automated image reader-based analysis of hemagglutination, such as the CypherOne™ HAI plate reader marketed by InDevR, Inc. (Boulder, CO, USA). Generally, agglutination is visually assessed by observing whether RBCs sink to the bottom of a suspension, indicating no agglutination, or agglutination inhibition, or form a diffuse network, indicating agglutination. If a container, such as a microtiter plate, has a round or U-shaped bottom, non-agglutinated RBCs can appear as a “dot” on the bottom of the container, while agglutinated RBCs are distributed more evenly in the suspension. In some embodiments, agglutination can be recorded, e.g., by photography and/or by digital imaging (e.g., by an automated image reader). If agglutination is not observed in a suspension containing a sample-virus mixture and human RBCs after a selected incubation time, then the biological sample is determined to contain antibodies that inhibit influenza virus HA glycoprotein hemagglutination activity.

An HAI assay described herein can be performed in any suitable container in which hemagglutination inhibition can be assessed. Preferably, a multi-well (e.g., 96-well), clear assay plate with round or U-shaped well bottoms, such as a 96-well U-bottom Microtiter™ plate marketed by Thermo Fisher Scientific (Waltham, MA, USA) is used. However, other formats and brands can be suitably used with appropriate adjustments to assay volumes and/or recordation/assessment of hemagglutination.

In some embodiments, replicates (e.g., duplicates) of each biological sample to be assessed can be subjected to an HAI assay disclosed herein. However, the disclosed assay provides surprisingly consistent results between samples that have been done in duplicate compared to just a single time.

In some embodiments, an HAI assay provided herein can include a step of determining the hemagglutination inhibition titer of the biological sample. Generally, the hemagglutination inhibition titer of the biological sample can be determined when a biological sample is serially diluted, and RBC agglutination is observed in more dilute samples while RBC agglutination is not observed in less dilute samples. Hemagglutination inhibition titer of the biological sample is determined as the highest dilution factor where RBC agglutination is not observed. Generally, hemagglutination inhibition titer of a biological sample is correlated with the concentration of antibodies that inhibit influenza virus hemagglutinin (HA) glycoprotein hemagglutination activity in the biological sample. It was discovered that an HAI assay described herein hemagglutination inhibition titer of a biological sample correlates well with influenza virus neutralizing antibody titer in the biological sample, as measured by a qualified microneutralization (MN) assay.

### Methods of Detecting Anti-SARS-COV-2 S IgG Antibodies

Provided herein are methods for determining if a biological sample contains antibodies that bind to a SARS-COV-2 Spike(S) glycoprotein, comprising: (i) obtaining a biological sample from a subject that has been simultaneously administered an immunogenic composition directed to a seasonal influenza virus and an immunogenic composition directed to a SARS-COV-2 virus; (ii) providing a surface coated with a SARS-COV-2 S glycoprotein; (iii) exposing the surface to the biological sample; (iv) exposing the surface to a secondary antibody; and (v) detecting the secondary antibody that is bound to the surface; wherein the biological sample contains antibodies that bind to the SARS-COV-2 S glycoprotein if secondary antibody is detected.

In embodiments, a SARS-COV-2 S glycoprotein on a surface can be a recombinant protein derived from a SARS-COV-2 virus spike glycoprotein or a SARS-COV-2 virus variant spike glycoprotein. In embodiments, the variant of SARS-COV-2 can be a B.1.1.7 SARS-COV-2 strain; a B.1.351 SARS-COV-2 strain; a P.1 SARS-COV-2 strain; a Cal.20C SARS-COV-2 strain; a B.1.617.2 SARS-COV-2 strain; a B.1.525 SARS-COV-2 strain; a B.1.526 SARS-COV-2 strain; a B.1.617.1 SARS-COV-2 strain; a C.37 SARS-COV-2 strain; a B.1.621 SARS-COV-2 strain; or a B.1.1.529 SARS-COV-2 strain. In embodiments, the SARS-COV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a SARS-COV-2 S glycoprotein from a SARS-COV-2 S omicron variant selected from the group consisting of: BA.1, BA.2.12.1, BA.2, BA.3, BA.4, BA.5, XBB.1.5, XBB.2.3, XBB.1.16, EG.5.1, JN.1, BQ.1.1, BF.7.

In some embodiments, a SARS-COV-2 S glycoprotein can have at least 90%, at least 92%, at least 94%, at least 95%, at least 96%, at least 97%, at least 98%, or at least 99% identity to SEQ ID NO: 1. In some embodiments, a SARS-COV-2 S glycoprotein can comprise or consist of any one of SEQ ID NOs: 1.

In embodiments, the SARS-COV-2 S glycoprotein has an inactive furin cleavage site. In embodiments, the inactive furin cleavage site comprises the amino acid sequence of QQAQ (SEQ ID NO: 3). In embodiments, amino acids 973 and 974 of the SARS-COV-2 S glycoprotein are proline, as compared to a wild-type SARS-COV-2 S glycoprotein having the amino acid sequence of SEQ ID NO: 1. In embodiments, a SARS-COV-2 S glycoprotein can include a transmembrane domain.

In some embodiments, a surface can be coated with more than one SARS-COV-2 S glycoprotein. For example, a surface can be coated with a SARS-COV-2 S glycoprotein derived from a first SARS-COV-2 virus or variant and a different SARS-COV-2 S glycoprotein derived from a second SARS-COV-2 virus or variant. In some embodiments, a surface can be coated with 3 or more different SARS-COV-2 S glycoproteins. In some embodiments, a surface can be coated with a SARS-COV-2 S glycoprotein comprising or consisting of SEQ ID NO: 4, a SARS-COV-2 S glycoprotein comprising or consisting of SEQ ID NO: 5, and/or a SARS-COV-2 S glycoprotein comprising or consisting of SEQ ID NO: 6.

In some embodiments, SARS-COV-2 S glycoprotein-coated surface can be treated to reduce or prevent non-specific binding to the SARS-COV-2 S glycoprotein and/or the surface by one or more components of a biological sample exposed thereto, reduce background noise during detection of the secondary antibody, and/or improve signal-to-noise ratio during detection of the secondary antibody. For example, a SARS-COV-2 S glycoprotein-coated surface can be treated with a blocking buffer prior to exposure to a biological sample. Suitable blocking buffers can include protein (e.g., bovine serum albumin, skim milk, or the like) or can be protein-free (e.g., detergent-base buffers), or a combination of protein and protein-free buffers. A SARS-COV-2 S glycoprotein-coated surface can be treated with a blocking buffer using any suitable conditions. For example, a blocking buffer can be incubated with a SARS-CoV-2 S glycoprotein-coated surface for 1 minute to about 12 hours (e.g., about 20 minutes to about 8 hours, about 30 minutes to about 4 hours, about 1 hour to about 3 hours, or about 1 to about 1.5 hours) at a temperature of from about 4° C. to about 40° C. (e.g., about 20° C. to about 30° C., or about 18° C. to about 27° C.).

A surface coated with a SARS-COV-2 S glycoprotein can be exposed to any suitable biological sample. In embodiments, the biological sample can be serum, plasma, blood, saliva, a nasopharyngeal swab, tissue or mucus. A biological sample can be used unmodified, or a biological sample can be diluted, concentrated, or otherwise suitably modified before exposure to a SARS-COV-2 S glycoprotein-coated surface. Preferably, a biological sample is a fluid sample or is processed to form a fluid so that any antibodies contained in the sample can associate with the SARS-COV-2 S glycoprotein-coated surface. In some embodiments, a biological sample can be stored as a frozen and/or dried sample prior to use in a method provided herein. Such samples are preferably reconstituted, diluted, and/or thawed prior to use.

A SARS-COV-2 S glycoprotein-coated surface can be exposed to a biological sample for any suitable time and in any suitable conditions to allow antibodies in the sample to bind to the SARS-COV-2 S glycoprotein. For example, a biological sample can be incubated with a SARS-COV-2 S glycoprotein-coated surface for 1 minute to about 12 hours (e.g., about 20 minutes to about 8 hours, about 30 minutes to about 4 hours, about 1 hour to about 3 hours, or about 2 hours). In some embodiments, a SARS-COV-2 S glycoprotein-coated surface can be exposed to a biological sample at a temperature of from about 4° C. to about 40° C. (e.g., about 20° C. to about 30° C., or about 18° C. to about 27° C.).

In some embodiments, a biological sample can be from a patient that has been administered one or more immunogenic composition comprising a SARS-COV-2 virus antigen and a seasonal influenza antigen. In embodiments, a method provided herein can be used to evaluate IgG response to an immunogenic composition designed to stimulate an immune response against a SARS-COV-2 virus and a seasonal influenza virus. Thus, methods, assays, and kits provided herein can be useful for rapid development of effective combination vaccines against emerging SARS-COV-2 variants and seasonal influenza virus strains.

In embodiments, antibodies in a biological sample that bind to a SARS-COV-2 S glycoprotein on a coated surface comprise IgG.

Typically, after exposing a biological sample to a SARS-COV-2 S glycoprotein on a surface, components of the biological sample that are not bound to the SARS-COV-2 S glycoprotein are removed. In some embodiments, unbound components can be washed away using, e.g., a buffer, such as phosphate-buffered saline (PBS) or PBS with detergent (e.g., Tween-20). A washing step can reduce or prevent unbound components from interfering with secondary antibody binding and/or non-specific enzyme activity during the detection step. In some embodiments, two or more washing steps can be performed after exposing a biological sample to a SARS-COV-2 S glycoprotein on a surface.

A secondary antibody is typically exposed to a biological sample-treated surface following washing of unbound sample components. A secondary antibody is selected to bind to a constant region of an antibody in the biological sample used. For example, a secondary antibody can bind to a constant region of a human IgA antibody (anti-human IgA) or a constant region of a human IgG antibody (anti-human IgG), if the biological sample is from a human. In some embodiments, the secondary antibody can comprise an anti-human IgG antibody.

In embodiments, the secondary antibody can be attached to a detectable tag. In embodiments, the tag comprises a chromogenic reporter (e.g., horseradish peroxidase or alkaline phosphatase), a fluorogenic reporter, an electrochemiluminescent reporter, or a quantitative PCR reporter.

In some embodiments, a secondary antibody is in a composition with one or more components to reduce or prevent non-specific binding of the secondary antibody and/or to reduce or prevent degradation of the secondary antibody or any included detectable tag. Suitable compositions include, for example, PBS or PBST (PBS with Tween-20).

A secondary antibody can be incubated with a biological sample-treated surface for any suitable time and in any suitable conditions to allow secondary antibodies to bind any antibodies from the biological sample. For example, a secondary antibody can be incubated with a biological sample-treated surface for 1 minute to about 24 hours (e.g., about 20 minutes to about 10 hours, about 30 minutes to about 4 hours, about 1 hour to about 3 hours, or about 1 hours) at a temperature of from about 4° C. to about 40° C. (e.g., about 20° C. to about 30° C., or about 18° C. to about 27° C.).

Typically, after exposing a secondary antibody to a biological sample-treated surface, excess secondary antibodies are removed. In some embodiments, unbound secondary antibodies can be washed away using, e.g., a buffer, such as phosphate-buffered saline (PBS) or PBS with detergent (e.g., Tween-20). In some embodiments, two or more washing steps can be performed after exposing a secondary antibody to a biological sample-treated surface. At this point, if the tested biological sample contained antibodies that bound to a SARS-COV-S glycoprotein-coated surface, secondary antibodies should be bound to the surface via the sample antibodies and the SARS-COV-S glycoproteins.

Detecting the secondary antibody that is bound to a surface can be done using any suitable method (e.g., colorimetric assay, fluorimetric assay, quantitative PCR, and the like). In some embodiments, a secondary antibody is detected using a signal molecule, either directly or indirectly. For example, if a secondary antibody includes a horseradish peroxidase (HRP) tag, a solution containing an HRP substrate (e.g., tetramethylbenzidene) is applied to the secondary antibody and presence of the HRP tag is detected by observing HRP-mediated color change. If an enzyme-based tag (e.g., HRP or alkaline phosphatase) is used on a secondary antibody, the reaction is typically stopped after sufficient time for the reaction to provide detectable signal (e.g., color change), but before non-specific reactions take place (e.g., oxidation) that result in a false positive. In some embodiments, an amount of time that an enzyme-based tag is allowed to react with a substrate can be selected to ensure that the amount of signal correlates with the amount of secondary antibody bound to the surface. Other detection methods (e.g., quantitative PCR-mediated detection of a quantitative PCR reporter, or label-free detection methods) can similarly be adjusted to provide correlation between signal strength and the amount of bound secondary antibodies. In such embodiments, a method provided herein can be used to quantitatively determine the amount of anti-SARS-COV-S glycoprotein antibodies were present in the tested biological sample. Thus, in some embodiments, a method provided herein can optionally include a step of quantitating anti-SARS-COV-S glycoprotein antibodies present in a biological sample.

In some embodiments, to facilitate quantitation of anti-SARS-COV-S glycoprotein antibodies present in a tested biological sample, a method can include the development of a standard curve using dilutions of one or more purified anti-SARS-COV-S glycoprotein antibodies (e.g., anti-SARS-COV-S glycoprotein IgG antibodies) to which signal from biological samples can be compared.

### Methods of Detecting Anti-SARS-COV-2 S Neutralizing Antibodies

Provided herein are methods for determining if a biological sample contains antibodies that neutralize a SARS-COV-2 virus, comprising: (i) obtaining the biological sample, wherein the biological sample is from a subject that has been simultaneously administered an immunogenic composition directed to a seasonal influenza virus and an immunogenic composition directed to a SARS-COV-2 virus; (ii) contacting the biological sample with a replication-deficient Maloney murine leukemia virus (MLV) pseudovirus to form a sample-pseudovirus mixture, the MLV pseudovirus comprising a recombinant SARS-COV-2 S (rS) glycoprotein and a gene of interest (GOI) encoding a reporter protein; (iii) adding cells expressing angiotensin converting enzyme 2 (ACE2) to the sample-pseudovirus mixture, wherein the cells are A549 or HEK293T cells; (iv) incubating the sample-pseudovirus mixture with the cells expressing ACE2 at 37° C., and quantifying expression of the reporter protein in the cells expressing ACE2 at 48 hours of incubation; (v) contacting control A549 or HEK293T cells expressing ACE2 with the MLV pseudovirus to form a virus-only control, wherein the MLV pseudovirus has not been contacted with the biological sample; and (vi) incubating the virus-only control with the control cells at 37° C., and quantifying expression of the reporter protein in the control cells expressing ACE2 at 48 hours of incubation; wherein if the expression of the reporter protein in the cells of step (iv) is less than expression of the reporter protein in the control cells of step (vi), then the biological sample contains neutralizing antibodies against the SARS-COV-2 virus.

In embodiments, a pseudovirus rS glycoprotein can have an amino acid sequence of a wild-type SARS-COV-2 S glycoprotein, such as from the Wuhan SARS-COV-2 strain, a B.1.1.7 SARS-COV-2 strain; a B.1.351 SARS-COV-2 strain; a P.1 SARS-COV-2 strain; a Cal.20C SARS-COV-2 strain; a B.1.617.2 SARS-COV-2 strain; a B.1.525 SARS-COV-2 strain; a B.1.526 SARS-COV-2 strain; a B.1.617.1; SARS-COV-2 strain; a C.37 SARS-COV-2 strain; aB.1.621 SARS-COV-2 strain; a B.1.1.529 SARS-COV-2 strain, or the like. A pseudovirus rS glycoprotein having a wild-type SARS-COV-2 S amino acid contains a furin cleavage site, RRAR (SEQ ID NO: 2), at positions 669-672 of the SARS-COV-2 S glycoprotein, as aligned (e.g., using BLAST) and numbered relative SEQ ID NO: 1. In some embodiments, a pseudovirus rS glycoprotein can have an amino acid sequence that is at least 90%, at least 92%, at least 94%, at least 95%, at least 96%, at least 97%, at least 98%, or at least 99% identity to a wild-type SARS-COV-2 S glycoprotein.

In some embodiments, a pseudovirus rS glycoprotein can have an inactive furin cleavage site. In some embodiments, the amino acid sequence of an inactive furin cleavage site can be QQAQ (SEQ ID NO: 3). In some embodiments, the amino acid sequence of an inactive furin cleavage site can be GG.

A replication-deficient Maloney murine leukemia virus (MLV) pseudovirus comprising SARS-COV-2 rS can be made by transfecting cells with: (i) a gene of interest GOI encoding a reporter protein (generally as part of a transfer plasmid); (ii) one or more plasmids encoding a Rev protein, a Tat protein, a Gag protein, a Pol protein; or a combination thereof; and (iii) a plasmid expressing a rS glycoprotein. In some embodiments, the cells comprise human epithelial cells (e.g., HEK293T (also called “293T”) cells or A549 cells).

A transfer plasmid containing a GOI (e.g., luciferase, green fluorescent protein, red fluorescent protein, or the like) used herein in a SARS-COV-2 pseudovirus is generally a replication-deficient DNA vector derived from a Maloney murine leukemia retrovirus. It contains the GOI, long terminal repeats (LTR) for integrating the GOI into a target cell's genome, and a packaging signal, but lacks sequences for expression of viral packaging proteins (Gag, Pol, and Env). In some embodiments, a transfer plasmid can comprise a Tat protein-encoding sequence to increase GOI expression in a target cell.

A replication-deficient MLV pseudovirus comprising a recombinant SARS-COV-2 S (rS) glycoprotein and a GOI encoding a reporter protein is contacted with a biological sample (e.g., human serum) to form a sample-pseudovirus mixture. In some embodiments, a biological sample can be heat-inactivated. In embodiments, heat inactivation comprises incubating the serum, plasma, or blood at a temperature of from about 37° C. to about 90° C. (e.g., about 40° C. to about 70° C., about 50° C. to about 60° C., or about 56° C.). In embodiments, heat inactivation comprises incubating the serum, plasma, or blood at a temperature of from about 37° C. to about 90° C. for from about 15 minutes to about 2 hours. In embodiments, the serum, plasma, or blood is incubated at about 37° C. to about 90° C. for about 30 minutes.

In some embodiments, a biological sample can be diluted prior to being combined with a replication-deficient MLV pseudovirus. For example, a biological sample can be diluted to reflect a predetermined SARS-COV-2 neutralizing antibody titer threshold (e.g., 1:2, 1:4, 1:5, 1:10, or the like). In some embodiments, a biological sample can be serially diluted two or more times (e.g., 2 to 50 times, 2 to 20 times, 2 to 15 times, 3, 4, 5, 6, 7, 8, 9, 10, 11, or 12 times). Serial dilution can be performed at any appropriate ratio (e.g., 1:2, 1:3, 1:4, 1:10, and the like). In some embodiments, a biological sample can first be diluted one or more times to reflect a predetermined SARS-COV-2 neutralizing antibody titer threshold (e.g., 1:4, 1:10, or the like), and then be further serially diluted (e.g., 1:2, 1:4, or the like). In some embodiments, a biological sample can be diluted in a buffer, such as Dulbeco's phosphate buffered saline (DPBS), optionally with calcium and/or magnesium.

A sample-pseudovirus mixture can be incubated for sufficient time to allow any neutralizing antibodies in the biological sample to bind the SARS-COV-2 rS protein of the replication-deficient MLV pseudovirus. In some embodiments, a sample-pseudovirus mixture can be incubated from 1 hour to about 24 hours, from 1 hour to about 12 hours, from 1 hour to about 8 hours, from 1 hour to about 6 hours, from 2 to about 6 hours, from 2 to about 7 hours, or from 2 to about 8 hours. In embodiments, a sample-pseudovirus mixture can be incubated from 1 day to about 7 days. In some embodiments, a sample-pseudovirus mixture can be incubated for 1 day, 2 days, 3 days, 4 days, 5 days, 6 days, or 7 days. In embodiments, a sample-pseudovirus mixture can be incubated for three days.

Following incubation, cells expressing ACE2 can be added to the sample-pseudovirus mixture and incubated. Sample-pseudovirus mixture and ACE2-expressing cells can be incubated for sufficient time to allow pseudovirus that has not been neutralized by antibodies in the biological sample to infect the ACE2-expressing cells and for the GOI to be expressed in infected cells. In some embodiments, a sample-pseudovirus mixture and ACE2-expressing cells can be incubated from 1 hour to about 24 hours, from 1 hour to about 12 hours, from 1 hour to about 8 hours, from 1 hour to about 6 hours, from 2 to about 6 hours, from 2 to about 7 hours, or from 2 to about 8 hours. In embodiments, a sample-pseudovirus mixture and ACE2-expressing cells can be incubated from 1 day to about 7 days. In some embodiments, a sample-pseudovirus mixture and ACE2-expressing cells can be incubated for 1 day, 2 days, 3 days, 4 days, 5 days, 6 days, or 7 days. In embodiments, a sample-pseudovirus mixture and ACE2-expressing cells can be incubated for three days.

Preferably, the ACE2-expressing cells comprise epithelial cells, such as A549 or HEK293T cells. Preferably, the ACE2-expressing cells are not in a monolayer prior to incubating with a sample-pseudovirus mixture.

A replication-deficient MLV pseudovirus comprising a recombinant SARS-COV-2 S (rS) glycoprotein and a GOI encoding a reporter protein used to perform a virus-only control by incubating the MLV pseudovirus as described above for the sample-pseudovirus mixture, except that no biological sample is contacted with the MLV pseudovirus. The MLV pseudovirus is then incubated with ACE2-expressing cells as described above.

Reporter protein expressed by the ACE2-expressing cells incubated with sample-pseudovirus is measured and compared to reporter protein expression in the ACE2-expressing cells from the virus-only control. If the expression of the reporter in the ACE2-expressing cells incubated with sample-pseudovirus is less than the expression of the reporter in the virus-only control, then the biological sample contains neutralizing antibodies against the SARS-COV-2 strain having an S glycoprotein with an amino acid sequence of the rS protein of the MLV pseudovirus.

In some embodiments, a biological sample can be contacted with two or more replication-deficient MLV pseudoviruses, each comprising a different SARS-COV-2 rS and a different reporter protein, simultaneously determining if a biological sample contains neutralizing antibodies against two or more SARS-COV-2 virus strains. For example, a biological sample can be contacted with a first replication-deficient MLV pseudovirus and a second replication-deficient MLV pseudovirus to form a sample-pseudovirus mixture, the first MLV pseudovirus comprising an rS glycoprotein from a first SARS-COV-2 virus strain (e.g., BA.5 strain) and a GOI encoding a first reporter protein (e.g., green fluorescent protein), and the second MLV pseudovirus comprising an rS glycoprotein from a second SARS-COV-2 virus strain (e.g., XBB.1.5 strain) and a GOI encoding a second reporter protein (e.g., red fluorescent protein). Such a method can determine whether a sample contains neutralizing antibodies to each of the SARS-COV-2 virus strains by measuring the relative expression of each of the different reporter proteins compared to the relevant virus-only control.

### Methods of Assessing or Selecting Immunogenic Compositions

Methods for assessing and/or selecting an immunogenic composition for use as a vaccine are also provided herein. Generally, methods for selecting an immunogenic composition for use as a vaccine include obtaining a plurality of biological samples from subjects that have been administered an immunogenic composition directed to a seasonal influenza virus and a SARS-COV-2 virus (e.g., from a medical study); performing one or more assay described above on the plurality of biological samples to determine antibody titers of the biological samples; and selecting the immunogenic composition for use as a vaccine if the antibody titers in the plurality of biological samples meets or exceeds a predetermined threshold for one or both of the seasonal influenza virus and SARS-COV-2 virus.

In some embodiments, the predetermined threshold can be based on the average antibody titers of a plurality of biological samples. In some embodiments, the predetermined threshold can be based on a certain percentage of samples within a plurality of samples exhibiting a particular antibody titer. In yet other embodiments, a predetermined threshold can be based on an increase in antibody titer following administration of an immunogenic composition as compared to antibody titer prior to administration of the immunogenic composition. For example, a predetermined threshold can be an average antibody titer following administration of an immunogenic composition that is more than two-fold (e.g., 3-fold, 4-fold, 5-fold, or the like) higher than the average hemagglutination inhibition titer prior to administration of the immunogenic composition.

In some embodiments, a method for selecting an immunogenic composition for use as a vaccine can comprise comparing antibody titers between biological samples from a population of subjects given one immunogenic composition and biological samples from a population of subjects given a different immunogenic composition, and selecting the immunogenic composition that results in higher antibody titers. In some embodiments, a method for selecting an immunogenic composition can comprise comparing three or more different immunogenic compositions.

In some embodiments, antibody titer need not be the only criteria used for selection of an immunogenic composition as a vaccine. Other selection criteria can also be considered, such as adverse effects, dosing, peak titer development time, cost, stability, and the like. Immunogenic compositions can differ in the type of antigen, presence or absence of an adjuvant, type of adjuvant, and the like.

### EXAMPLES

### Example 1—HAI Assay Validation

A hemagglutination inhibition (HAI) assay is developed using human red blood cells (RBCs) and either egg-derived influenza virus or virus-like particles (VLPs) as the agglutinin. The assay is tested and validated using human serum samples.

### Materials and Methods

### Seasonal Influenza Viruses and VLPs

The validation of HAI assay for egg-derived influenza viruses is performed using the following four vaccine-homologous 2019-2020 Northern Hemisphere seasonal strains: A/Kansas/14/2017 (A/H3N2), A/Brisbane/02/2018 (A/H1N1), B/Maryland/15/2016 (B/Victoria Lineage), and B/Phuket/3073/2013 (B/Yamagata Lineage). VLPs were produced in Sf9 insect cells infected with baculoviruses bearing cloned genes of influenza HA, neuraminidase (NA), and M1. The membrane-bound VLPs were approximately 100 nM in diameter and morphologically resembled influenza virus particles; the NA is enzymatically active. The VLP HAI assay validation included four recombinant Baculovirus/Sf9-produced VLPs corresponding to the aforementioned homologous seasonal influenza vaccine strains and four VLPs corresponding to the following four antigenically drifted (heterologous) A (H3N2) viruses: A/California/94/2019, A/Cardiff/0508/2019, A/Netherlands/1268/2019, and A/Tokyo/EH1801/2018.

### Human RBCs

Human RBCs (Type O) with K3EDTA as an anticoagulant (Biological Specialty Corporation/BioIVT, Westbury, NY, USA, cat. HUMANRBK3-0101896) were used for the HAI assay. Each new donor's erythrocytes were screened for comparability using a panel of previously tested sera, at least 80% of which must return titers within 2-fold of the prior values for acceptance of the new donor. Each washed and diluted lot of 0.75% red blood cell suspension is tested with negative and positive quality control sera to confirm consistent behavior before use in the assay.

### Test Serum Samples

The serum samples used in the assay validation experiments were from healthy humans (BioIVT, Westbury, NY, USA) self-reporting receipt of the influenza vaccine within 1 year before sample collection, human sera that showed positive HAI during the pre-validation serum screening processes, and HA-specific sheep serum samples from virus HA-hyperimmune sheep obtained from the National Institute for Biological Standards and Control and Novavax influenza vaccine non-clinical studies. Negative serum is HA antibody-depleted/stripped human serum (Valley Biomedical, Winchester, VA, USA, cat. HS1200W) diluted 1:2 in PBS. The depletion of HA antibodies from human sera is performed using a negative selection by HA-cross-linked agarose beads (by Novavax, Inc., Gaithersburg, MD, USA).

### HAI Assay Procedure

The HAI assay is conducted per the WHO Manual for the laboratory diagnosis and virology surveillance of influenza, with some modifications (such as human RBCs and the automated assay readout method mentioned below). To remove nonspecific inhibitors of HA, the serum samples were incubated at 37° C. for 18 to 20 h with a receptor-destroying enzyme (RDE; Denka Seiken Co., Ltd., San Jose, CA, USA, cat. 370013) diluted 1:4 in Dulbecco's Phosphate Buffered Saline with calcium and magnesium (DPBS; Quality Biological, Gaithersburg, MD, USA, cat. 114-059-101), followed by heat-inactivation at 56° C. for 30±2 min and further diluted to 1:10 with DPBS. In the HAI assay, each RDE-treated sample is diluted 1:2 serially in a 96-well U-bottom Microtiter™ plate (Thermo Fisher Scientific, Waltham, MA, USA, cat. 2205) for a total of 10 dilutions with DPBS. The viral stock is adjusted to 4 hemagglutination (HAg) units in DPBS with 80 nM of oseltamivir to prevent possible NA-mediated agglutination (NA inhibitor; ChemScene, Monmouth Junction, NJ, USA, cat. CS-0553). After incubating with 4 HAg units of the virus, the sample-virus mixture is then incubated with 0.75% human RBCs (BioIVT, cat. HUMANRBK3-0101896) for 80 to 100 min at room temperature (RT). To determine serum antibody titers, the HAI assay plates were scored using the automated and validated CypherOne™ Hemagglutination Analyzer (InDevR Inc., Boulder, CO, USA, software versions 3.2.0.0 and 4.0.0.19). The HAI titers were determined from the reciprocal of the highest serum sample dilution that completely prevented hemagglutination.

### Validation Parameters: Precision, Specificity, Linearity, Sensitivity, Assay Robustness, and Sample Stability

The assay parameters evaluated for each vaccine-homologous strain of egg-derived virus and VLP agglutinins were precision, specificity, linearity, sensitivity (lower limit of quantitation [LLOQ]), robustness, and sample stability. The intra-, inter-, and total-assay precision were tested at both the individual sample and strain levels, representing the overall assay variance of all samples tested for each egg-derived virus/VLP strain. Strain-specific sheep sera were used to test the ability of the HAI assay to measure and differentiate the influenza virus type- and subtype-specific antibodies (anti-A/H1N1 [A strain subtype 1 HA], anti-A/H3N2 [A strain subtype 3 HA], and anti-B strain HA antibodies). Negative human serum samples included in the specificity runs were expected to consistently test negative (geometric mean titer [GMT] of 5-7). To determine the assay linearity, for each strain, two influenza HAI-positive samples were tested undiluted and further serially diluted in 1:2 dilution series (from 1:2 up to 1:256) in negative serum, with a target of a minimum of six dilutions above the LLOQ. Samples were tested in replicates in a total of six runs by two analysts over 3 days. The expected HAI titers at each dilution were calculated from the overall HAI GMT from all runs of the undiluted sample divided by the dilution factor. The accuracy of the linearity at each dilution is evaluated in terms of percent relative bias, where values of 100% or −50% correspond to a titer that is 2-fold higher or 2-fold lower, respectively, than the expected titer.

The assay sensitivity is determined in terms of the LLOQ, which is the lowest HAI titer with acceptable precision and accuracy. The LLOQ is determined for samples assayed in the linearity tests. Precision (percent geometric coefficient of variation [% GCV] of HAI titers) and accuracy (percent relative bias) were estimated across the runs for these samples.

Robustness is tested for all egg-derived virus/VLP strains to evaluate the effect of RBC age (storage time from receipt/collection time), serum-agglutinin incubation time, and serum-agglutinin-RBC incubation time (plate reading time) on the HAI results. For the RBC suspension storage time robustness assay, the panel of serum samples were tested using 0.75% RBC stored at 2 to 8° C. for 2 weeks (14±3 days) and 0.75% RBC prepared from 10% RBC suspension stored at 2 to 8° C. for 2 weeks (14±3 days). The HAI GMT of the test storage conditions is compared with the overall HAI GMT from the precision assay runs (utilizing fresh 0.75% RBCs stored at 2 to 8° C. for less than a week [≤7 days]; baseline).

Plate reading time robustness is determined at 75, 90, 120, and 150 min after the addition of the 0.75% RBC suspension to the serum-virus mixture (incubated for 60 min). The HAI GMTs were compared with the standard 90-min reading time. To evaluate the combined serum-agglutinin incubation time and plate reading time robustness, serum-agglutinin incubation time is varied (50, 60, and 70 min) and, for each duration of incubation, the plate reading time is varied (75, 90, 120, and 150 min) after the addition of RBCs. Baseline HAI GMTs were from assay runs in which serum-agglutinin incubation time is 60 min and the plate reading time is 90 min after RBC addition. Additionally, for egg-derived virus/VLP titration and back-titration (virus/VLP titration plate reading time robustness), the plate reading time is determined at 90±10 min following RBC addition.

The stability of RDE-treated samples stored at 2 to 8° C. (1 and 2 months), ≤−20° C. (2 months), and subjected to two freeze/thaw cycles is assessed and compared with the overall HAI GMT from precision runs (baseline; with samples stored at 2 to 8° C. for ≤7 days). The stability of neat (undiluted) serum samples stored at −20±10° C. (1 month) and subjected to seven freeze/thaw cycles (from storage in a −80±10° C. freezer) is assessed using three quality control (QC) samples for each egg-derived virus/VLP strain. The results were compared with the overall GMT from the precision runs (baseline) as described above.

### Singleton (Single Titer) Testing of Serum Samples in the Influenza VLP HAI Assay

To analyze the influence of singleton testing on individual samples (single titer, i.e., singlicate), the validation data of the VLP HAI assay for the four homologous seasonal vaccine strains were used and reproduced. The GMT and % GCV were determined for the validation dataset of each vaccine-homologous strain. For each serum sample, the GMT and % GCV were determined for (1) singleton titers (N=24), (2) paired replicate (duplicate) GMT (N=12), and (3) random titer replicates (duplicate) GMT (N=12). Random titer pairings were intended to simulate potential titer pairings based on the data collected in the validation studies.

The percentage difference in GMT for randomly chosen replicates (random replicate 1 and random replicate 2) is determined relative to the GMT of paired replicates as shown in Equation 1 below.

$$\% Difference = 100 \times \frac{( Random HAI GMT - Paired HAI GMT )}{Paired HAI GMT} \qquad Equation 1$$

For assessing the influence of singleton results and paired duplicates on precision (% GCV), 36 serum samples were tested for the four vaccine-homologous strains. The % difference for % GCV of singleton titers and random replicates is calculated relative to the % GCV of paired replicates as shown in Equation 2 below.

$$\% Difference = 100 \times \frac{( Paired or Random \% GCV - Singleton \% GCV )}{Singleton \% GCV} \qquad Equation 2$$

For evaluating the influence of singleton testing on clinical study results, HAI anti-body titer data corresponding to VLPs from the Phase 3 clinical study qNIV-E-301 (Shinde, V.; Cho, I.; Plested, J. S.; Agrawal, S.; Fiske, J.; Cai, R.; Zhou, H.; Pham, X.; Zhu, M.; Cloney-Clark, S.; et al. Comparison of the Safety and Immunogenicity of a Novel Matrix-M-Adjuvanted Nanoparticle Influenza Vaccine with a Quadrivalent Seasonal Influenza Vaccine in Older Adults: A Randomized Controlled Trial. Lancet Infect. Dis. 2022, 22, 73-84) were utilized. Aside from the aforementioned four vaccine-homologous seasonal influenza strains and the four heterologous viruses, this clinical study included three additional heterologous wild-type strains (A/South Australia/34/2019, A/Idaho/13/2018, and B/is hington/02/2019). Antibody titers using VLP HAI obtained in duplicate (n=2) from approximately 1286 test subjects and two study protocol arms (2019-2020 Fluzone® Quadrivalent and Quad-NIV) were used to model the influence of reporting single titer (n=1) results (randomly selected from one of the paired duplicates) instead of the GMT of paired duplicate (n=2) values. The per-protocol population included 1279 to 1286 test subjects (two time points: Day 0 [pre-vaccination] and Day 28 [post-vaccination]; two vaccination groups: 2019-2020 Fluzone Quadrivalent and Quad-NIV). The GMT (by strain and vaccine group) is calculated as the reported GMT (duplicate) values of the antibody titers using wild-type sequence VLP-HAI. The results of GMT calculated using paired replicates (duplicates) were compared with the GMT calculated using individual titers that were randomly selected. The randomly selected titer GMT results were determined twice (Random titers 1 and 2). The geometric mean ratio (GMR; post-/pre-vaccination) were determined using the reported GMT from paired duplicate samples and randomly selected single titers. The random titer pairings included were intended to simulate potential titer pairings based on the data collected in clinical studies.

The percent seroprotection rate (% SPR) is determined using data from the study qNIV-E-301. The percentage of results with titer ≥1:40 is determined using the reported GMT from paired duplicate samples and using randomly selected single titers (Random 1 and Random 2) for subject visits on Day 28. The percent seroconversion rate (% SCR) is also determined using qNIV-E-301 study data. The results (%) for subject visits on Day 0 (unvaccinated) and Day 28 (vaccinated) where the titer or GMT on Day 28 is higher by ≥4-fold were determined using the reported GMT from paired duplicate samples and using randomly selected single titers (Random 1 and Random 2).

### Correlation Analysis of the HAI Assay and the MN Assay

Serum samples were assessed in the HAI assay (using the same methodology as for validation) and in a qualified influenza MN assay for each indicated wild-type strain (A/Brisbane/02/2018, A/Kansas/14/2017, and B/Maryland/15/2016). The results from the final titers of both assays were compared to determine the correlation between them.

### Statistical Analysis

Statistical analyses of the validation results were performed using SAS® version 9.4 (SAS Institute Inc., Cary, NC, USA) in a Windows (Microsoft Corp., Redmond, WA, USA) environment. The intra- and inter-assay precision were assessed by determining the % GCV through the variance component analysis with the sample as a fixed effect and analyst and day as random effects. The correlation between the HAI and MN assay results is determined by performing linear regression analysis using GraphPad Prism® software version 9.3.1 (GraphPad Software, San Diego, CA, USA).

### Results

All four seasonal influenza homologous virus strains, using either egg-derived virus or VLP, met the acceptance criteria of intra-, inter-, and total-assay precision≤50% GCV for at least 80% of the samples tested for each strain. Additionally, assays were shown to be specific, linear, robust, sensitive with an LLOQ of 10.

### Discussion

The data presented herein describes the development and validation of a novel robust HAI assay using egg-derived viruses/recombinant VLPs with human RBCs to assess the immunogenicity of combined SARS-COV-2 and seasonal influenza vaccines in clinical trial settings. Validation of the HAI method ensures the reliability and reproducibility of results.

### Conclusions

In conclusion, the validated egg-derived virus and VLP HAI assays with human RBCs performed similarly with comparable precision and accuracy, indicating the suitability for evaluating humoral immune response (HAI) for seasonal influenza in human serum samples from clinical studies on combined SARS-COV-2 and seasonal influenza vaccines.

### Example 2—SARS-COV-2 Pseudovirus Assay Validation

We validated a pseudovirus based neutralization assay for evaluating the immunogenicity of combined SARS-COV-2 and seasonal influenza vaccines. The assay is a BSL-2 based assay, which is cost effective, leads to a rapid turnaround, and enables evaluation of immunogenicity of combined SARS-COV-2 and seasonal influenza vaccines (e.g., neutralizing antibody production) against emerging SARS-COV-2 variants. This assay is a surrogate for live virus neutralization assay and does not require use of a BSL-3 laboratory.

### Methods

This assay is suitable for measurement of neutralization ability in clinical samples (serum from human subjects immunized with combined COVID-19 and seasonal influenza vaccines). This assay may be used to evaluate the ability of combined SARS-COV-2/seasonal influenza vaccines to stimulate an immune response against SARS-COV-2 viruses (e.g., Wuhan strain, BA.1 strain, BA.2 strain, BA.5 strain, and the like).

SARS-COV-2 pseudoviruses expressing recombinant SARS-COV-2 spike proteins were produced by co-transfecting cells with plasmids encoding MLV retroviral backbone expressing luciferase reporter, plasmids encoding non-surface proteins for retroviral production and plasmid expressing SARS-COV-2 spike protein under the control of a mammalian promoter.

SARS-COV-2 pseudoviruses were produced by transfecting HEK293T cells with (i) a plasmid encoding a MLV transfer plasmid encoding a reporter protein (e.g., luciferase or ZsGreen); (ii) retroviral helper plasmids; and (iii) a plasmid expressing a SARS-COV-2 S glycoprotein. The transfer plasmid included the reporter protein under the control of a cytomegalovirus (CMV) promoter. Pseudoviruses were harvested from the cell culture supernatant.

Pseudoviruses were then incubated from serum of subjects that were immunized with a combined SARS-COV-2/quadrivalent influenza vaccine. As a control, pseudoviruses were incubated with cell culture medium without serum (virus-only controls).

The pseudoviruses were used to infect cells expressing the ACE2 receptor and incubated at 37° C. for 48 to 72 hours. As a negative control, cells were infected with cell culture medium in the absence of pseudovirus. The luminescence of the cells (from luciferase) was measured. A reduction of luciferase signal indicated a neutralization of infection.

The dose dependence of luminescence on pseudovirus level was determined by infecting cells expressing ACE2 receptor with different amounts of pseudovirus, and detecting luciferase luminescence (RLU) 2 days post-infection. Controls were cells infected but not treated with serum (virus control), and cells without pseudovirus (cell control). Percent (%) inhibition/neutralization of pseudovirus infection by the serum relative to virus control was determined.

Pseudovirus-based neutralization titer using different amounts of pseudovirus was assessed to optimize the assay procedure. The kinetics of the pseudovirus neutralization assay endpoint was evaluated by measuring luminescence at 48 and 72 hours, and comparing the signal to background (SIB) ratios.

### Assay Quality-Precision

Quality control (QC) samples were tested by 2 operators (analysts) on 2 different days, and duplicates of each sample were tested in the same assay. For prototype/Wuhan strain, high-, medium-, and low-quality QC samples were used. For variants, 2 or 3 QC samples were used.

### Assay Quality-Linearity

One sample was serially diluted (6 times, 4-fold dilution series), and evaluated in duplicate by 2 different operators.

Correlation Analyses with Live Virus-Based Microneutralization (MN) Assay

Samples were tested in a validated live virus MN assay (360biolabs) (n=13), followed by comparison of data with from the pseudovirus neutralization assay using linear regression analysis.

Correlation with Anti-S IgG Antibodies and hACE2 Binding Inhibition

Samples were tested in a validated anti-S IgG assay (n=15) or validated hACE2 binding inhibition assay (n=8), followed by comparison of data with pseudovirus neutralization assay results using linear regression analysis.

### Example 3—SARS-COV-2 IgG Assay Validation

An ELISA assay is developed. Microtiter assay plate (Thermo Fisher Scientific, Waltham, MA, USA) well surfaces were coated with recombinant SARS-COV-2 S (rS) protein (modified Wuhan spike glycoprotein (SEQ ID NO: 4)) by direct adsorption of a 1.5-1.6 μg/ml spike glycoprotein solution in phosphate buffered saline (PBS) for 15 to 48 hours at 2-8° C. A range of 16 to 48 hours is found to produce consistent results. Plates were then washed with phosphate-buffered saline with Tween 20 (PBST) and blocked for 1-1.5 hours with blocking buffer (Thermo Fisher Scientific).

Reference standards (IgG anti-SARS-COV-2 S protein antibodies) diluted in pooled human saliva were used to develop a standard curve against which IgG antibody content in biological samples is compared to determine IgG content.

Samples were then added to the wells, allowing anti-rS protein IgG antibodies to bind to rS protein coated on plate (2±10 mins hours of incubation).

The plates were again washed with PBST, and then a goat anti-human IgG secondary antibody conjugated with horseradish peroxidase (HRP; from Invitrogen) is added and incubated for 1 hour±10 mins at room temperature.

A final wash step is performed, followed by addition of 3,3′5,5′-tetramethylbenzidine substrate (TMB, from Thermo Fisher Scientific). The reaction is stopped after 20-25 minutes by TMB stop solution (ScyTek Laboratories, Logan, UT, USA).

The optical density (OD) of the chromogenic signal is directly proportional to the amount of anti-rS IgG captured on the plate, providing a quantifiable measurement of IgG concentration in the serum sample. Anti-spike IgG concentration is calculated by interpolating to the levels of the IgG reference standard curve.

1. A method for determining if a biological sample contains antibodies that inhibit influenza virus hemagglutinin (HA) glycoprotein hemagglutination activity, comprising:
a. obtaining the biological sample, wherein the biological sample is from a subject that has been simultaneously administered an immunogenic composition directed to a seasonal influenza virus and an immunogenic composition directed to a SARS-COV-2 virus;
b. reducing or removing non-specific inhibitors of HA from the biological sample to produce a treated sample;
c. exposing the treated sample to an influenza virus having an HA glycoprotein to produce a sample-virus mixture;
d. incubating the sample-virus mixture with human red blood cells (RBCs), the human RBCs being at a concentration of about 0.5% to about 1%, or about 0.75%; and
e. detecting agglutination of the human RBCs;
wherein the biological sample contains antibodies that inhibit HA glycoprotein hemagglutination activity if agglutination of the RBCs is not detected.

2. The method of claim 1, wherein the biological sample is serially diluted, and each serial dilution of the sample is subjected to steps b, c, d, and e.

3. The method of claim 2, comprising determining the hemagglutination inhibition titer of the biological sample.

4. The method of claim 1, wherein the virus comprises an egg-derived influenza virus, or a virus-like particle (VLP).

5. A method for determining if a biological sample contains antibodies that neutralize a SARS-COV-2 virus, comprising:
a. obtaining the biological sample, wherein the biological sample is from a subject that has been simultaneously administered an immunogenic composition directed to a seasonal influenza virus and an immunogenic composition directed to a SARS-COV-2 virus;
b. contacting the biological sample with a replication-deficient Maloney murine leukemia virus (MLV) pseudovirus to form a sample-pseudovirus mixture, the MLV pseudovirus comprising a recombinant SARS-COV-2 S (rS) glycoprotein and a gene of interest (GOI) encoding a reporter protein;
c. adding cells expressing angiotensin converting enzyme 2 (ACE2) to the sample-pseudovirus mixture, wherein the cells are A549 or HEK293T cells;
d. incubating the sample-pseudovirus mixture with the cells expressing ACE2 at 37° C., and quantifying expression of the reporter protein in the cells expressing ACE2 at 48 hours of incubation;
e. contacting control A549 or HEK293T cells expressing ACE2 with the MLV pseudovirus to form a virus-only control, wherein the MLV pseudovirus has not been contacted with the biological sample; and
f. incubating the virus-only control with the control cells at 37° C., and quantifying expression of the reporter protein in the control cells expressing ACE2 at 48 hours of incubation;
wherein if the expression of the reporter protein in the cells of step d is less than expression of the reporter protein in the control cells of step f, then the biological sample contains neutralizing antibodies against the SARS-COV-2 virus.

6. A method for simultaneously determining if a biological sample contains neutralizing antibodies against two or more SARS-COV-2 virus strains, comprising:
a. obtaining the biological sample, wherein the biological sample is from a subject that has been simultaneously administered an immunogenic composition directed to a seasonal influenza virus and an immunogenic composition directed to a SARS-COV-2 virus;
b contacting the biological sample with a first replication-deficient MLV pseudovirus and a second replication-deficient MLV pseudovirus to form a sample-pseudovirus mixture, the first MLV pseudovirus comprising an rS glycoprotein from a first SARS-COV-2 virus strain and a GOI encoding a first reporter protein, and the second MLV pseudovirus comprising an rS glycoprotein from a second SARS-COV-2 virus strain and a GOI encoding a second reporter protein;
c. adding cells expressing ACE2 to the sample-pseudovirus mixture, wherein the cells are A549 or HEK293T cells;
d. incubating the sample-pseudovirus mixture with the cells expressing ACE2 at 37° C., and quantifying expression of the first reporter protein and the second reporter protein in the cells expressing ACE2 at 48 hours of incubation;
e. contacting control A549 or HEK293T cells expressing ACE2 with the first MLV pseudovirus and the second MLV pseudovirus to form a virus-only control, wherein the first MLV pseudovirus and the second MLV pseudovirus have not been contacted with the biological sample; and
f. incubating the virus-only control with the control cells at 37° C., and quantifying expression of the first reporter protein and the second reporter protein in the control cells expressing ACE2 at 48 hours of incubation;
wherein if the expression of the first reporter protein in the cells of step d is less than expression of the first reporter protein in the control cells of step f, then the biological sample contains neutralizing antibodies against the first SARS-COV-2 virus strain, and
wherein if the expression of the second reporter protein in the cells of step d is less than expression of the second reporter protein in the control cells of step f, then the biological sample contains neutralizing antibodies against the second SARS-COV-2 virus strain.

7. The method of claim 6, wherein the first reporter protein is red fluorescent protein (RFP) and the second reporter protein is green fluorescent protein (GFP).

8. A method for determining if a biological sample contains antibodies that bind to a SARS-COV-2 Spike(S) glycoprotein, comprising:
a. obtaining the biological sample, wherein the biological sample is from a subject that has been simultaneously administered an immunogenic composition directed to a seasonal influenza virus and an immunogenic composition directed to a SARS-COV-2 virus;
b providing a surface coated with a recombinant SARS-COV-2 S (rS) glycoprotein;
c. exposing the surface to the biological sample;
d. exposing the surface to a secondary antibody; and
e. detecting the secondary antibody that is bound to the surface;
wherein the biological sample contains antibodies that bind to the rS glycoprotein if the secondary antibody is detected.

9. The method of claim 8, wherein the rS glycoprotein on the coated surface has at least 90% identity to SEQ ID NO: 1.

10. The method of claim 8, wherein the rS glycoprotein on the coated surface:
a. comprises an inactive furin cleavage site; and/or
b, wherein amino acids 973 and 974 of the SARS-COV-2 S glycoprotein are proline, as compared to a wild-type SARS-COV-2 S glycoprotein having the amino acid sequence of SEQ ID NO: 1.

11. The method of claim 8, wherein the antibodies that bind to the rS glycoprotein on the coated surface comprise IgG.

12. The method of claim 11, wherein the secondary antibody is an anti-IgG antibody.

13. The method of claim 1, wherein the immunogenic composition directed to the seasonal influenza virus and the immunogenic composition directed to the SARS-COV-2 virus are a single immunogenic composition.

14. The method of claim 1, wherein the immunogenic composition directed to the seasonal influenza virus comprises an antigen from each of at least three different seasonal influenza viruses.

15. The method of claim 5, wherein the immunogenic composition directed to the seasonal influenza virus and the immunogenic composition directed to the SARS-COV-2 virus are a single immunogenic composition.

16. The method of claim 5, wherein the immunogenic composition directed to the seasonal influenza virus comprises an antigen from each of at least three different seasonal influenza viruses.

17. The method of claim 6, wherein the immunogenic composition directed to the seasonal influenza virus and the immunogenic composition directed to the SARS-COV-2 virus are a single immunogenic composition.

18. The method of claim 6, wherein the immunogenic composition directed to the seasonal influenza virus comprises an antigen from each of at least three different seasonal influenza viruses.

19. The method of claim 8, wherein the immunogenic composition directed to the seasonal influenza virus and the immunogenic composition directed to the SARS-COV-2 virus are a single immunogenic composition.

20. The method of claim 8, wherein the immunogenic composition directed to the seasonal influenza virus comprises an antigen from each of at least three different seasonal influenza viruses.
